(19) United States
(12) Patent Application Publication
(10) Pub. No.: US 2024/0280525 A1
(43) Pub. Date: Aug. 22, 2024

(54) GAS IDENTIFICATION METHOD, AND GAS IDENTIFICATION SYSTEM

(71) Applicant: Panasonic Intellectual Property Management Co., Ltd., Osaka (JP)

(72) Inventor: Toshiki NIINOMI, Osaka (JP)

(21) Appl. No.: 18/570,435

(22) PCT Filed: Jul. 5, 2022
(86) PCT No.: PCT/JP2022/026769

§ 371 Date: Dec. 14, 2023

(30) Foreign Application Priority Data
Jul. 9, 2021 (JP) 2021-114292

(51) Int. Cl. G01N 27/12; G01N 33/00

(52) U.S. Cl. CPC G01N 27/121; G01N 33/0006; G01N 33/0034

(57) ABSTRACT

A gas identification method includes: (a) obtaining a signal outputted from an odor sensor exposed to a sample gas during a predetermined measurement period; (b) extracting a feature of the signal obtained in (a); (c) obtaining humidity data indicating a humidity of the sample gas; (d) correcting the feature extracted in (b), based on the humidity data obtained in (c); and (e) identifying the sample gas by using a trained model for identifying the sample gas, based on the feature corrected in (d), and outputting an identification result.

### TECHNICAL FIELD

The present disclosure relates to a gas identification method and a gas identification system.

### BACKGROUND ART

A gas identification method of extracting a feature of a signal outputted from a sensor exposed to a sample gas and identifying the sample gas based on the feature has been known. As an example of such a gas identification method, Patent Literature (PTL) 1 discloses a technique of identifying an analyte by using, as a feature, the kurtosis, strength ratio, wavelength, strength and the like of a pulse signal outputted when the analyte is detected.

### CITATION LIST

### Patent Literature

[PTL 1] WO2018/207524

### SUMMARY OF INVENTION

### Technical Problem

However, such a conventional gas identification method has a problem that the identification accuracy for a sample gas is reduced due to moisture in the sample gas.

In view of this, the present disclosure provides a gas identification method and a gas identification system that can improve the identification accuracy for a sample gas.

### Solution to Problem

A gas identification method according to an aspect of the present disclosure uses a sensor that outputs a signal according to an adsorption concentration of a gas. The gas identification method includes: (a) obtaining a signal outputted from the sensor exposed to a sample gas during a predetermined measurement period; (b) extracting a feature of the signal obtained in (a); (c) obtaining humidity data indicating a humidity of the sample gas; (d) correcting the feature extracted in (b), based on the humidity data obtained in (c); and (e) identifying the sample gas by using a trained model for identifying the sample gas, based on the feature corrected in (d), and outputting an identification result.

Moreover, a gas identification system according to an aspect of the present disclosure includes: a sensor that outputs a signal according to an adsorption concentration of a gas; an exposer that exposes the sensor to a sample gas during a predetermined measurement period; a signal obtainer that obtains a signal outputted from the sensor during the predetermined measurement period; an extractor that extracts a feature of the signal obtained by the signal obtainer; a humidity data obtainer that obtains humidity data indicating a humidity of the sample gas; a corrector that corrects the feature extracted by the extractor, based on the humidity data obtained by the humidity data obtainer; and an identifier that identifies the sample gas by using a trained model for identifying the sample gas, based on the feature corrected by the corrector, and outputs an identification result.

Furthermore, a gas identification method according to an aspect of the present disclosure uses a sensor that outputs a signal according to an adsorption concentration of a gas. The gas identification method includes: (a) obtaining a signal outputted from the sensor exposed to a sample gas having a certain humidity; (b) extracting a feature of the signal obtained in (a); (c) generating, from the feature extracted in (b), a pseudo data set indicating a feature corresponding to the sample gas having a humidity other than the certain humidity, based on a predetermined correction coefficient; and (d) outputting, as a training data set to be used in a trained model for identifying the sample gas, the pseudo data set generated in (c).

It should be noted that general or specific aspects of the present disclosure may be realized as a system, a method, an integrated circuit, a computer program, a computer readable recording medium such as a Compact Disc Read Only Memory (CD-ROM), or any given combination thereof.

### Advantageous Effects of Invention

A gas identification method or the like according to an aspect of the present disclosure can improve the identification accuracy for a sample gas.

### BRIEF DESCRIPTION OF DRAWINGS

FIG. 1 is a block diagram illustrating the configuration of a gas identification system according to Embodiment 1.

FIG. 2 is a schematic diagram illustrating an example of an exposer of the gas identification system according to Embodiment 1.

FIG. 3 is a flowchart illustrating operations performed by the gas identification system according to Embodiment 1.

FIG. 4 is a graph illustrating an example of a temporal change in the value of a signal outputted from an odor sensor according to Embodiment 1.

FIG. 5 is a diagram for describing the detail of step S107 in the flowchart of FIG. 3.

FIG. 6 is a diagram for describing the detail of step S107 in the flowchart of FIG. 3.

FIG. 7 is a table showing the result of an experiment for confirming an effect achieved by the gas identification system according to Embodiment 1.

FIG. 8 is a conceptual diagram for describing a first correction function and a second correction function according to Variation 1 of Embodiment 1.

FIG. 9 is a block diagram illustrating the configuration of a gas identification system according to Embodiment 2.

FIG. 10 is a flowchart illustrating operations performed by the gas identification system according to Embodiment 2.

FIG. 11 is a conceptual diagram for describing operations performed by the gas identification system according to Embodiment 2.

FIG. 12 is a table showing the result of an experiment for confirming an effect achieved by the gas identification system according to Embodiment 2.

FIG. 13 is a table showing the result of an experiment for confirming an effect achieved by the gas identification system according to Embodiment 2.

### DESCRIPTION OF EMBODIMENTS

(Technique 1) A gas identification method uses a sensor that outputs a signal according to an adsorption concentration of a gas. The gas identification method includes: (a) obtaining a signal outputted from the sensor exposed to a sample gas during a predetermined measurement period; (b) extracting a feature of the signal obtained in (a); (c) obtaining humidity data indicating a humidity of the sample gas; (d) correcting the feature extracted in (b), based on the humidity data obtained in (c); and (e) identifying the sample gas by using a trained model for identifying the sample gas, based on the feature corrected in (d), and outputting an identification result.

According to Technique 1, the feature is corrected based on the humidity data, and the sample gas is identified by using the trained model for identifying the sample gas, based on the feature corrected. Accordingly, the identification accuracy for the sample gas can be improved while reducing the influence of the moisture in the sample gas.

(Technique 2) The gas identification method according to Technique 1. In (d), the feature extracted in (b) is corrected by using a correction function representing a relationship between the feature extracted in (b), the humidity data obtained in (c), and a reference humidity that is a humidity of the sample gas learned by the trained model.

According to Technique 2, the feature is corrected to the level of the learned humidity by using the correction function, and therefore the feature corrected can be approximated to a feature that is a training data set learned by the trained model even when the humidity data indicates an unlearned humidity that is different from the learned humidity (=reference humidity). Accordingly, the identification accuracy for the sample gas can be improved by inputting, to the trained model, the feature corrected by using the correction function.

(Technique 3) The gas identification method according to Technique 2. The correction function is represented by Y=X+A(H₀−H) where A represents a correction coefficient calculated from a gradient of a linear function by which a function representing a relationship between the humidity of the sample gas and the feature of the sample gas is approximated, X represents the feature extracted in (b), H represents the humidity data obtained in (c), H₀ represents the reference humidity, and Y represents a correction value of the feature extracted in (b).

According to Technique 3, the correction function can be easily determined.

(Technique 4) The gas identification method according to Technique 2 or 3. The predetermined measurement period includes at least a first period and a second period, the correction function includes at least a first correction function specific to the first period and a second correction function specific to the second period, in (b), a first feature of the signal outputted from the sensor exposed to the sample gas during the first period and a second feature of the signal outputted from the sensor exposed to the sample gas during the second period are extracted, and in (d), the first feature extracted in (b) is corrected by using the first correction function and the second feature extracted in (b) is corrected by using the second correction function.

The amount of moisture in the sample gas adsorbed by the sensor gradually increases from the start of exposure to the sample gas. Such a temporal change in the amount of moisture adsorbed by the sensor makes the feature of the signal outputted from the sensor during the first period and the feature of the signal outputted from the sensor during the second period different from each other. According to Technique 4, by using the first correction function specific to the first period and the second correction function specific to the second period, the features can be corrected with high accuracy while suppressing the influence of the temporal change in the amount of moisture adsorbed by the sensor.

(Technique 5) The gas identification method according to Technique 2 or 3. The sensor includes at least a first sensor and a second sensor, the correction function includes at least a first correction function specific to the first sensor and a second correction function specific to the second sensor, in (a), a first signal outputted from the first sensor exposed to the sample gas during the predetermined measurement period and a second signal outputted from the second sensor exposed to the sample gas during the predetermined measurement period are obtained, in (b), a first feature of the first signal obtained in (a) and a second feature of the second signal obtained in (a) are extracted, and in (d), the first feature extracted in (b) is corrected by using the first correction function and the second feature extracted in (b) is corrected by using the second correction function.

According to Technique 5, even when the first odor sensor and the second odor sensor are different in characteristics (e.g., responsivity to humidity or the like), the features can be corrected with high accuracy by using the correction functions each of which is optimal for a corresponding one of the first odor sensor and the second odor sensor.

(Technique 6) The gas identification method according to Technique 2 or 3. The feature includes a value of the signal and a gradient of the signal that have changed due to the sensor being exposed to the sample gas, the correction function includes a first correction function specific to the value of the signal and a second correction function specific to the gradient of the signal, in (b), the value of the signal and the gradient of the signal are extracted as the feature of the signal, and in (d), the value of the signal is corrected by using the first correction function and the gradient of the signal is corrected by using the second correction function.

According to Technique 6, even when the sensitivity and gradient of the signal are different in responsivity to humidity, the features can be corrected with high accuracy by using the correction functions each of which is optimal for a corresponding one of the features.

(Technique 7) The gas identification method according to any one of Techniques 1 to 5. In (b), the feature responsive to humidity is extracted.

According to Technique 7, when a feature that is not responsive to humidity is corrected, there is a possibility that the identification accuracy for the sample gas is reduced. Therefore, the identification accuracy for the sample gas can be improved by excluding a feature that is not responsive to humidity from a target to be corrected.

(Technique 8) The gas identification method according to Technique 7. The feature includes at least one of a value of the signal or a gradient of the signal that have changed due to the sensor being exposed to the sample gas.

According to Technique 8, the identification accuracy for the sample gas can be improved by extracting, as the feature responsive to humidity, at least one of the value or gradient of the signal.

(Technique 9) The gas identification method according to any one of Techniques 1 to 8. In (a), the signal outputted from the sensor is obtained via a network.

According to Technique 9, even when the sensor is located at a remote place, the signal outputted from the sensor can be easily obtained via a network.

(Technique 10) The gas identification method according to any one of Techniques 1 to 9 further includes (f) generating a training data set to be used in the trained model based on the feature extracted in (b), and outputting the training data set generated.

According to Technique 10, the identification accuracy for the sample gas can be further improved.

(Technique 11) The gas identification method according to Technique 10. In (f), a plurality of training data sets each of which corresponds to a different one of a plurality of humidities of the sample gas are generated, and the plurality of training data sets generated are outputted.

According to Technique 11, the identification accuracy for the sample gas can be further improved.

(Technique 12) A gas identification system includes: a sensor that outputs a signal according to an adsorption concentration of a gas; an exposer that exposes the sensor to a sample gas during a predetermined measurement period; a signal obtainer that obtains a signal outputted from the sensor during the predetermined measurement period; an extractor that extracts a feature of the signal obtained by the signal obtainer; a humidity data obtainer that obtains humidity data indicating a humidity of the sample gas; a corrector that corrects the feature extracted by the extractor, based on the humidity data obtained by the humidity data obtainer; and an identifier that identifies the sample gas by using a trained model for identifying the sample gas, based on the feature corrected by the corrector, and outputs an identification result.

According to Technique 12, the corrector corrects the feature, based on the humidity data, and the identifier identifies the sample gas by using the trained model for identifying the sample gas, based on the feature corrected. Accordingly, the identification accuracy for the sample gas can be improved while reducing the influence of the moisture in the sample gas.

(Technique 13) A gas identification method uses a sensor that outputs a signal according to an adsorption concentration of a gas. The gas identification method includes: (a) obtaining a signal outputted from the sensor exposed to a sample gas having a certain humidity; (b) extracting a feature of the signal obtained in (a); (c) generating, from the feature extracted in (b), a pseudo data set indicating a feature corresponding to the sample gas having a humidity other than the certain humidity, based on a predetermined correction coefficient; and (d) outputting, as a training data set to be used in a trained model for identifying the sample gas, the pseudo data set generated in (c).

According to Technique 13, the pseudo data set indicating the feature corresponding to the sample gas having a humidity other than the certain humidity is generated from the feature extracted, based on the predetermined correction coefficient, and the pseudo data set generated is outputted as a training data set to be used in the trained model for identifying the sample gas. Thus, the trained model can be trained using two or more levels, and the identification accuracy for the sample gas can be improved.

It should be noted that general or specific aspects of the present disclosure may be realized as a system, a method, an integrated circuit, a computer program, a computer readable recording medium such as a CD-ROM, or any given combination thereof.

Hereinafter, embodiments are specifically described with reference to the Drawings.

It should be noted that each of the embodiments described below shows a general or specific example of the present disclosure. The numerical values, shapes, materials, constituent elements, arrangement and connection of the constituent elements, steps, order of the steps etc. shown in the following embodiments are mere examples, and therefore do not limit the present disclosure. Moreover, among the constituent elements in the following embodiments, constituent elements not recited in any one of the independent claims are described as arbitrary constituent elements.

### Embodiment 1

### 1-1. Configuration of Gas Identification System

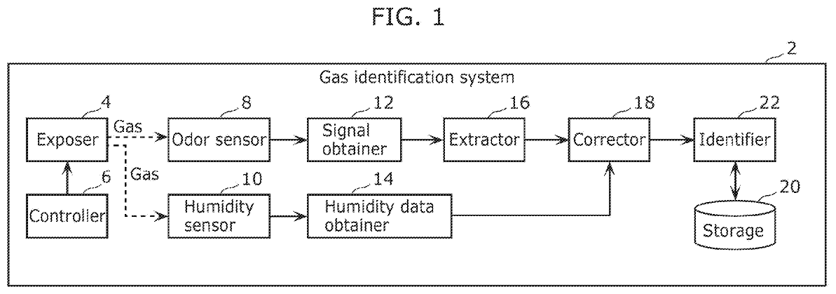

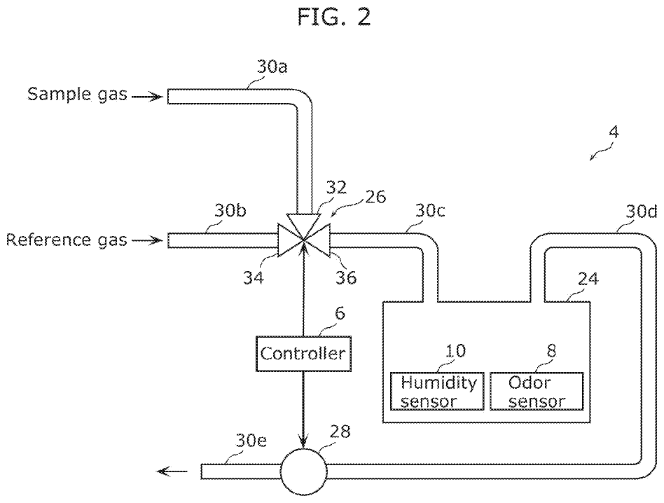

First, the configuration of gas identification system 2 according to Embodiment 1 is described with reference to FIG. 1 and FIG. 2. FIG. 1 is a block diagram illustrating the configuration of gas identification system 2 according to Embodiment 1. FIG. 2 is a schematic diagram illustrating an example of exposer 4 of gas identification system 2 according to Embodiment 1.

As illustrated in FIG. 1, gas identification system 2 includes exposer 4, controller 6, odor sensor 8 (an example of a sensor), humidity sensor 10, signal obtainer 12, humidity data obtainer 14, extractor 16, corrector 18, storage 20, and identifier 22.

Gas identification system 2 identifies a sample gas based on a feature of a signal outputted from odor sensor 8 exposed to the sample gas. Examples of the sample gas include a gas collected from food, exhaled breath obtained from a human body, air around a human body, and air obtained from a room in a building.

In the present embodiment, gas identification system 2 is used to identify the odor of the sample gas. In other words, gas identification system 2 identifies which of a plurality of odorants is included in the sample gas.

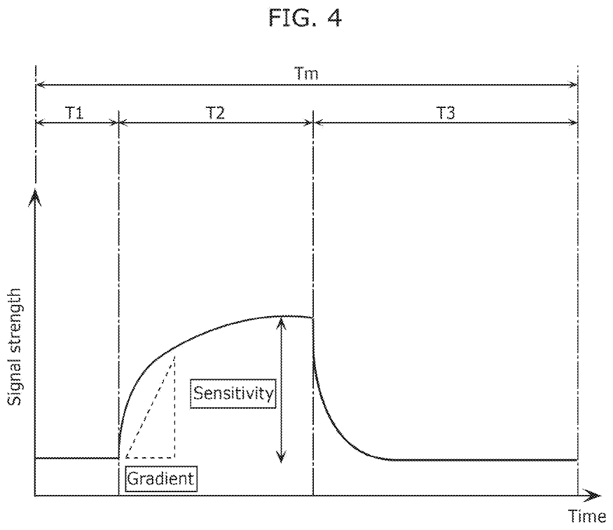

Exposer 4 is a mechanism for exposing odor sensor 8 to the sample gas. Specifically, exposer 4 exposes odor sensor 8 to the sample gas only during second period T2 (an example of a predetermined measurement period) in measurement period Tm that consists of first period T1, second period T2 subsequent to first period T1, and third period T3 subsequent to second period T2 (see FIG. 4 to be described later). Moreover, exposer 4 exposes odor sensor 8 to a reference gas only during first period T1 and third period T3 in measurement period Tm.

The reference gas is a gas that is a reference for measurement and is, for example, a gas including no odorant or the like. Specific examples of the reference gas include an inert gas such as air and nitrogen, and a gas obtained by removing a chemical substance from a sample gas by a filter or the like.

Here, the specific configuration of exposer 4 is described with reference to FIG. 2. As illustrated in FIG. 2, exposer 4 includes housing 24, three-way electromagnetic valve 26, pump 28, and pipes 30a, 30b, 30c, 30d, and 30e.

Housing 24 is a box-shaped container for housing odor sensor 8 and humidity sensor 10. As described later, the sample gas or the reference gas is introduced into housing 24.

Three-way electromagnetic valve 26 is an electromagnetic valve for switching a gas to be introduced into housing 24, and is driven by controller 6. Three-way electromagnetic valve 26 includes first inlet port 32, second inlet port 34, and outlet port 36. Three-way electromagnetic valve 26 can be switched between a first state in which first inlet port 32 and outlet port 36 communicate with each other and a second state in which second inlet port 34 and outlet port 36 communicate with each other. In the first state, first inlet port 32 and outlet port 36 are open and second inlet port 34 is closed. On the other hand, in the second state, second inlet port 34 and outlet port 36 are open and first inlet port 32 is closed.

First inlet port 32 communicates with, via pipe 30a, a sample gas supply source (not illustrated) for supplying the sample gas. Second inlet port 34 communicates with, via pipe 30b, a reference gas supply source (not illustrated) for supplying the reference gas. Outlet port 36 communicates with housing 24 via pipe 30c.

Pump 28 is a suction pump for introducing the sample gas or the reference gas into housing 24 and discharging, from housing 24, the sample gas or reference gas introduced, and is driven by controller 6. Pump 28 communicates with housing 24 via pipe 30d and also communicates with an exhaust duct (not illustrated) via pipe 30e.

Three-way electromagnetic valve 26 is switched to the first state and the first state is maintained during second period T2 in measurement period Tm while pump 28 is driven. In this case, the sample gas supplied from the sample gas supply source is introduced into housing 24 via pipe 30a, first inlet port 32 and outlet port 36 of three-way electromagnetic valve 26, and pipe 30c. Thus, odor sensor 8 is exposed to the sample gas introduced into housing 24. Moreover, the sample gas introduced into housing 24 is discharged to the exhaust duct via pipe 30d, pump 28, and pipe 30e.

Furthermore, three-way electromagnetic valve 26 is switched to the second state and the second state is maintained during first period T1 and third period T3 in measurement period Tm while pump 28 is driven. In this case, the reference gas supplied from the reference gas supply source is introduced into housing 24 via pipe 30b, second inlet port 34 and outlet port 36 of three-way electromagnetic valve 26, and pipe 30c. Thus, odor sensor 8 is exposed to the reference gas introduced into housing 24. Since odor sensor 8 exposed to the reference gas outputs a signal during first period T1 and third period T3 in measurement period Tm, the signal that is a reference corresponding to the surrounding environment can be obtained even if the surrounding environment is changed for every measurement. Moreover, the reference gas introduced into housing 24 is discharged to the exhaust duct via pipe 30d, pump 28, and pipe 30e.

With reference to FIG. 1, controller 6 controls the driving of each of three-way electromagnetic valve 26 and pump 28 of exposer 4. Specifically, controller 6 drives pump 28 and switches three-way electromagnetic valve 26 to the second state, and the second state is maintained during first period T1 and third period T3 in measurement period Tm. Moreover, controller 6 drives pump 28 and switches three-way electromagnetic valve 26 to the first state, and the first state is maintained during second period T2 in measurement period Tm.

Odor sensor 8 is located inside housing 24 of exposer 4 and exposed to a gas introduced into housing 24 to thereby output a signal corresponding to the adsorption concentration of the gas. For example, odor sensor 8 is an electrically resistant sensor. Specifically, odor sensor 8 includes a sensing element made of a sensing film, and a pair of electrodes electrically connected to the sensing element. The electrical resistance value of the sensing element varies depending on the adsorption concentration of an odorant of a gas adsorbed by the sensing element. Odor sensor 8 outputs, to signal obtainer 12 via the pair of electrodes, a signal corresponding to the electrical resistance value of the sensing element, as a voltage signal or current signal. It should be noted that odor sensor 8 is not limited to an electrically resistant sensor and examples of odor sensor 8 may include various sensors such as an electrochemical sensor, a semiconductor sensor, a field-effect transistor sensor, a surface acoustic wave sensor, a quartz crystal sensor, and the like.

Gas identification system 2 may include a plurality of the above-described odor sensors 8. In this case, the plurality of odor sensors 8 are arranged in an array inside housing 24. Sensing elements of the plurality of odor sensors 8 may be made of mutually different materials. In this case, the sensing elements made of mutually different materials show mutually different adsorption behaviors to the same chemical substance (odorant or the like). Therefore, the plurality of odor sensors 8 output mutually different signals with respect to the same chemical substance. Accordingly, mutually different features can be extracted from the signals outputted from the plurality of odor sensors 8, and thus the identification accuracy for the sample gas can be improved.

Humidity sensor 10 is disposed inside housing 24 of exposer 4 and detects the humidity of the sample gas introduced into housing 24 during second period T2 in measurement period Tm. Humidity sensor 10 outputs, to humidity data obtainer 14, humidity data indicating the humidity of the sample gas detected.

Signal obtainer 12 obtains a signal outputted from odor sensor 8 during second period T2 in measurement period Tm and outputs, to extractor 16, the signal obtained.

Humidity data obtainer 14 obtains the humidity data outputted from humidity sensor 10 during second period T2 in measurement period Tm and outputs, to corrector 18, the humidity data obtained.

Extractor 16 extracts, from the signal obtained by signal obtainer 12, a feature of the signal. Specifically, extractor 16 extracts, as the feature, the maximum value (hereinafter, referred to as “sensitivity”) or gradient (the amount of change in the value of a signal per unit time) of the signal that have changed due to odor sensor 8 being exposed to the sample gas, for example. Extractor 16 outputs, to corrector 18, the feature extracted. It should be noted that extractor 16 may extract a plurality of features from the signal obtained by signal obtainer 12. In this case, extractor 16 extracts, as the plurality of features, both the sensitivity and gradient of the signal, for example.

Corrector 18 corrects the feature extracted by extractor 16, based on the humidity data obtained by humidity data obtainer 14. Specifically, corrector 18 corrects the feature extracted by extractor 16 by using a correction function representing the relationship between the feature extracted by extractor 16, the humidity data obtained by humidity data obtainer 14, and a reference humidity that is the humidity of a sample gas learned by a trained model (to be described later). Corrector 18 outputs, to identifier 22, the feature corrected.

Storage 20 is memory that stores the trained model used by identifier 22. The trained model is a logical model for identifying the sample gas. Specifically, for example, the trained model is a logical model for identifying which of odorants is included in the sample gas. For example, the trained model uses, as an input, the feature corrected by corrector 18 and outputs information indicating which of odorants is included in the sample gas. A training data set used in the trained model is, for example, a feature of a signal outputted from odor sensor 8 exposed to the sample gas having the humidity of 40% (hereinafter, also referred to as “learned humidity”). It should be noted that the trained model may output information indicating whether an odorant is included in the sample gas.

For example, the trained model is built by machine learning using, as a labeled training data set, a known odorant and a feature of a signal outputted from odor sensor 8 exposed to the sample gas including the known odorant. For example, a neural network, a random forest, a support vector machine, a self-organizing map, or the like is used for building a logical model in machine learning.

Identifier 22 identifies the sample gas by using the trained model stored in storage 20, based on the feature corrected by corrector 18. Specifically, identifier 22 identifies which of odorants is included in the sample gas, by using the trained model. For example, identifier 22 outputs, to a display (not illustrated) or the like provided in gas identification system 2, information indicating an identification result. Thus, the identification result outputted by identifier 22 is displayed on the display.

### 1-2. Operations Performed by Gas Identification System

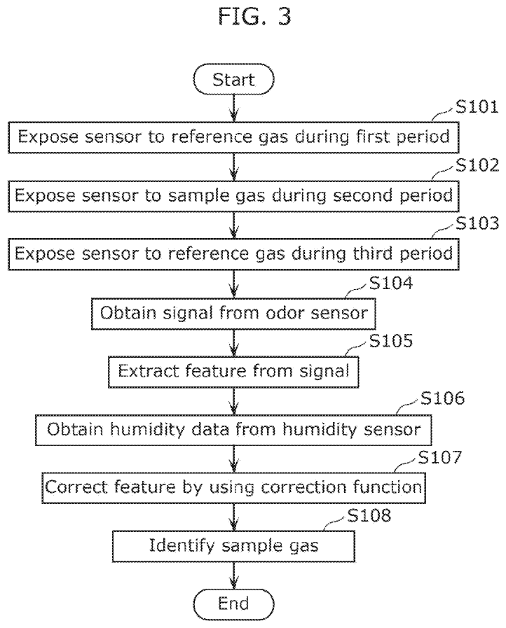

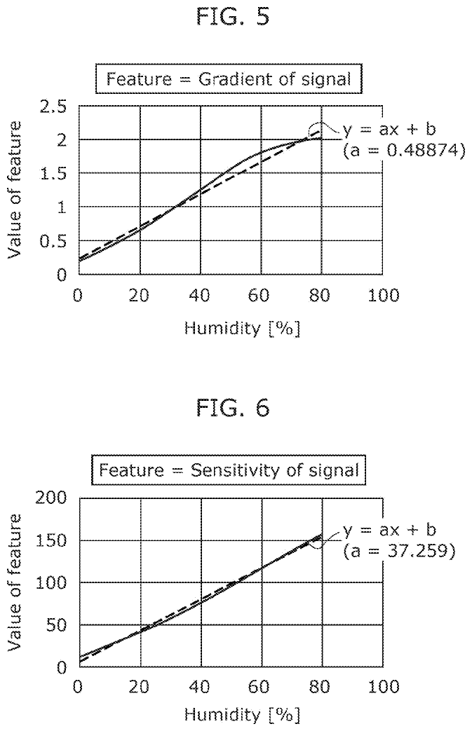

Next, operations performed by gas identification system 2 according to Embodiment 1 are described with reference to FIG. 3 to FIG. 6. FIG. 3 is a flowchart illustrating a flow of operations performed by gas identification system 2 according to Embodiment 1. FIG. 4 is a graph illustrating an example of a temporal change in the value of a signal outputted from odor sensor 8 according to Embodiment 1. Each of FIG. 5 and FIG. 6 is a diagram for describing the detail of step S107 in the flowchart of FIG. 3.

As illustrated in FIG. 3, controller 6 drives pump 28 and switches three-way electromagnetic valve 26 to the second state, and the second state is maintained during first period T1 in measurement period Tm. Thus, a reference gas is introduced into housing 24 and odor sensor 8 is exposed to the reference gas (S101).

Next, controller 6 drives pump 28 and switches three-way electromagnetic valve 26 to the first state, and the first state is maintained during second period T2 in measurement period Tm. Thus, a sample gas is introduced into housing 24 and odor sensor 8 is exposed to the sample gas (S102).

Next, controller 6 drives pump 28 and switches three-way electromagnetic valve 26 to the second state, and the second state is maintained during third period T3 in measurement period Tm. Thus, the reference gas is introduced into housing 24 and odor sensor 8 is exposed to the reference gas (S103).

During measurement period Tm, the value (signal strength) of the signal outputted from odor sensor 8 changes temporally, as illustrated in FIG. 4, for example. Specifically, during first period T1 in which odor sensor 8 is exposed to the reference gas, the value of the signal outputted from odor sensor 8 is maintained at an almost constant value (hereinafter, referred to as “reference value”). Next, during second period T2 in which odor sensor 8 is exposed to the sample gas, a sensing element of odor sensor 8 adsorbs the sample gas (specifically, an odorant included in the sample gas), and thus the value of the signal outputted from odor sensor 8 is increased. Next, during third period T3 in which odor sensor 8 is exposed to the reference gas again, the sample gas (specifically, the odorant included in the sample gas) is removed from the sensing element of odor sensor 8, and thus the value of the signal outputted from odor sensor 8 is decreased to the reference value.

It should be noted that first period T1, second period T2, and third period T3 are appropriately set according to the type of odor sensor 8 or the like. First period T1 is, for example, at least one second and not more than 10 seconds. Second period T2 is, for example, at least five seconds and not more than 30 seconds. Third period T3 is, for example, at least 10 seconds and not more than 100 seconds.

After that, signal obtainer 12 obtains the signal outputted from odor sensor 8 during second period T2 in measurement period Tm (S104) and outputs the signal obtained to extractor 16. Extractor 16 extracts a feature from the signal obtained by signal obtainer 12 (S105) and outputs the feature extracted to corrector 18. Specifically, extractor 16 extracts, as the feature, the sensitivity or gradient of the signal as illustrated in FIG. 4, for example.

Humidity data obtainer 14 obtains humidity data outputted from humidity sensor 10 during second period T2 in measurement period Tm (S106) and outputs the humidity data obtained to corrector 18.

Corrector 18 corrects, by using a correction function, the feature extracted by extractor 16 to the level of a learned humidity (S107) and outputs, to identifier 22, the feature corrected. Here, the correction function is explained. The correction function is represented by Equation 1 below, where A represents a correction coefficient, X represents the feature extracted by extractor 16, H represents the humidity data obtained by humidity data obtainer 14, H₀ represents the reference humidity, and Y represents a correction value of the feature extracted by extractor 16.

$$Y = X + A ( {H}_{0} - H ) \qquad ( Equation 1 )$$

It should be noted that correction coefficient A is calculated from the gradient of a linear function by which a function representing the relationship between the humidity and feature of the sample gas is approximated using the least-squares method. Hereinafter, how to calculate correction coefficient A is described.

When the feature is the gradient of the signal, the relationship between the humidity of the sample gas and the gradient of the signal is a proportional relationship as illustrated by the solid line in the graph in FIG. 5. In other words, the gradient of the signal is a feature responsive to humidity. It should be noted that it is assumed that the relationship between the humidity of the sample gas and the gradient of the signal illustrated in FIG. 5 is obtained in advance. Then, as illustrated by the broken line in the graph in FIG. 5, a function representing the relationship between the humidity of the sample gas and the gradient of the signal is approximated by a linear function (y=ax+b) using the least-squares method. The value obtained by dividing gradient a (=0.48874) of the linear function by the humidity of 20% is determined as correction coefficient A (=a/20%).

Likewise, when the feature is the sensitivity of the signal, the relationship between the humidity of the sample gas and the sensitivity of the signal is a proportional relationship as illustrated by the solid line in the graph in FIG. 6. In other words, the sensitivity of the signal is a feature responsive to humidity. It should be noted that it is assumed that the relationship between the humidity of the sample gas and the sensitivity of the signal illustrated in FIG. 6 is obtained in advance. Then, as illustrated by the broken line in the graph in FIG. 6, a function representing the relationship between the humidity of the sample gas and the sensitivity of the signal is approximated by a linear function (y=ax+b) using the least-squares method. The value obtained by dividing gradient a (=37.259) of the linear function by the humidity of 20% is determined as correction coefficient A (=a/20%).

Next, an example of a method for correcting a feature using the correction function of Equation 1 is described. Hereinafter, an example will be described in which reference humidity H₀ is 40%, that is, the humidity of the sample gas learned by the trained model is 40%.

When humidity data H is 0% and H₀=40%, H=0%, and A=a/20% are substituted in the correction function of Equation 1, Y=X+2a is obtained. In other words, corrector 18 corrects feature X extracted by extractor 16 to the level of the humidity of 40%, by adding “+2a” to feature X, using the correction function.

Moreover, when humidity data H is 20% and H₀=40%, H=20%, and A=a/20% are substituted in the correction function of Equation 1, Y=X+a is obtained. In other words, corrector 18 corrects feature X extracted by extractor 16 to the level of the humidity of 40%, by adding “+a” to feature X, using the correction function.

Furthermore, when humidity data H is 40% and H₀=40%, H=40%, and A=a/20% are substituted in the correction function of Equation 1, Y=X is obtained. In other words, corrector 18 adds “0” to feature X extracted by extractor 16, using the correction function. In this case, corrector 18 does not substantially correct feature X.

Furthermore, when humidity data H is 60% and H₀=40%, H=60%, and A=a/20% are substituted in the correction function of Equation 1, Y=X−a is obtained. In other words, corrector 18 corrects feature X extracted by extractor 16 to the level of the humidity of 40%, by adding “−a” to feature X, using the correction function.

Furthermore, when humidity data H is 80% and H₀=40%, H=80%, and A=a/20% are substituted in the correction function of Equation 1, Y=X−2a is obtained. In other words, corrector 18 corrects feature X extracted by extractor 16 to the level of the humidity of 40%, by adding “−2a” to feature X, using the correction function.

With reference to the flowchart in FIG. 3, after step S107, identifier 22 identifies the sample gas by using the trained model stored in storage 20, based on the feature corrected by corrector 18 (S108). After that, the flowchart in FIG. 3 ends.

### 1-3. Effect

The gradient and sensitivity of the signal are features responsive to humidity and easily influenced by the moisture included in the sample gas. Specifically, when the feature is the gradient of the signal, the relationship between the humidity of the sample gas and the gradient of the signal is a proportional relationship as illustrated by the solid line in the graph in FIG. 5. Moreover, when the feature is the sensitivity of the signal, the relationship between the humidity of the sample gas and the sensitivity of the signal is a proportional relationship as illustrated by the solid line in the graph in FIG. 6.

Therefore, when humidity data H is an unlearned humidity that is different from a learned humidity (40%, for example), feature X extracted by extractor 16 differs from a feature that is a training data set learned by the trained model. Accordingly, when feature X extracted by extractor 16 is inputted as it is to the trained model, there is a problem that the identification accuracy for the sample gas is reduced.

In contrast, in the present embodiment, corrector 18 corrects the feature extracted by extractor 16 to the level of the learned humidity, by using the correction function of Equation 1. Thus, the feature corrected by corrector 18 can be approximated to the feature that is the training data set learned by the trained model even when humidity data H is an unlearned humidity that is different from the learned humidity. Accordingly, the identification accuracy for the sample gas can be improved by inputting, to the trained model, the feature corrected by corrector 18.

### 1-4. Example and Comparative Example

The following experiment was carried out for confirming the above-described effect. In this experiment, nitrogen was used as a reference gas. Moreover, as sample gases, five sample gases A, B, C, D, and E (A to E) with five types of reference odors for odor determination (odor intensity level 2) were used. Sample gases A to E were gases each of which includes a different one of the five types of chemical compounds below.
Sample gas A: β-phenylethyl alcohol (odor of flower)
Sample gas B: Methylcyclopentenolone (odor of something sweet and burnt)
Sample gas C: Isovaleric acid (odor of sweaty socks)
Sample gas D: γ-Undecalactone (odor of ripe fruit)
Sample gas E: skatole (odor of mold)

As a comparative example, signals outputted from an odor sensor were obtained by exposing the odor sensor to sample gases A to E (at the temperature of 23° C.) having humidities of 0%, 20%, 40%, 60%, and 80%. A signal outputted from the odor sensor was obtained 100 times for each of sample gases A to E having the humidity of 0%. Moreover, a signal outputted from the odor sensor was obtained 100 times for each of sample gases A to E having the humidity of 20%. Furthermore, a signal outputted from the odor sensor was obtained 100 times for each of sample gases A to E having the humidity of 40%. Furthermore, a signal outputted from the odor sensor was obtained 100 times for each of sample gases A to E having the humidity of 60%. Furthermore, a signal outputted from the odor sensor was obtained 100 times for each of sample gases A to E having the humidity of 80%. A test for identifying a sample gas was performed by inputting, to a trained model, features extracted from the signals as they were. It should be noted that the learned humidity learned by the trained model was 40%.

Moreover, as an example, signals outputted from an odor sensor were obtained by exposing the odor sensor to sample gases A to E (at the temperature of 23° C.) having humidities of 0%, 20%, 40%, 60%, and 80%. A signal outputted from the odor sensor was obtained 100 times for each of sample gases A to E having the humidity of 0%. Moreover, a signal outputted from the odor sensor was obtained 100 times for each of sample gases A to E having the humidity of 20%. Furthermore, a signal outputted from the odor sensor was obtained 100 times for each of sample gases A to E having the humidity of 40%. Furthermore, a signal outputted from the odor sensor was obtained 100 times for each of sample gases A to E having the humidity of 60%. Furthermore, a signal outputted from the odor sensor was obtained 100 times for each of sample gases A to E having the humidity of 80%. A test for identifying a sample gas was performed by correcting, using the correction function of Equation 1, features extracted from the signals, and inputting, to a trained model, the features corrected. It should be noted that the learned humidity learned by the trained model was 40%.

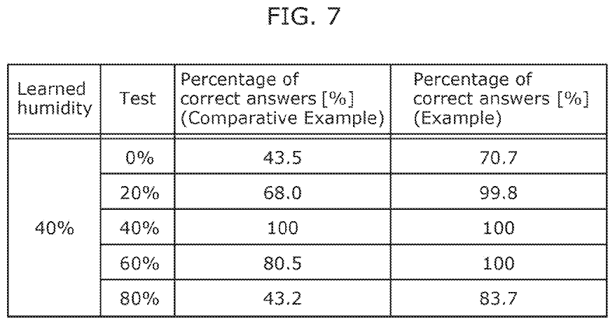

The result of the experiment is shown in FIG. 7. FIG. 7 is a table showing the result of the experiment for confirming an effect achieved by gas identification system 2 according to Embodiment 1. As shown in FIG. 7, in the comparative example, the percentages of correct answers given by the trained model were 43.5% for sample gases A to E having the humidity of 0%, 68.0% for sample gases A to E having the humidity of 20%, 100% for sample gases A to E having the humidity of 40%, 80.5% for sample gases A to E having the humidity of 60%, and 43.2% for sample gases A to E having the humidity of 80%.

In contrast, in the example, the percentages of correct answers given by the trained model were 70.7% for sample gases A to E having the humidity of 0%, 99.8% for sample gases A to E having the humidity of 20%, 100% for sample gases A to E having the humidity of 40%, 100% for sample gases A to E having the humidity of 60%, and 83.7% for sample gases A to E having the humidity of 80%.

Thus, it was confirmed that, even when the humidities (0%, 20%, 60%, and 80%) of sample gases A to E were different from the learned humidity, the percentages of correct answers given by the trained model in the example was increased compared to those in the comparative example.

### 1-5. Variation 1

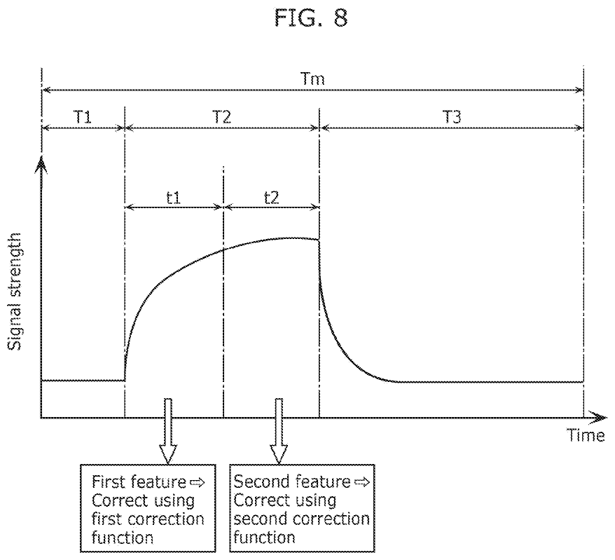

Although corrector 18 uses a single correction function specific to second period T2 in measurement period Tm in the present embodiment, the present disclosure is not limited to this example. FIG. 8 is a conceptual diagram for describing a first correction function and a second correction function according to Variation 1 of Embodiment 1.

In the present variation, second period T2 in measurement period Tm includes first period t1 and second period t2. Extractor 16 extracts a first feature of a signal outputted from odor sensor 8 exposed to a sample gas during first period t1 and a second feature of a signal outputted from odor sensor 8 exposed to the sample gas during second period t2.

Corrector 18 corrects the first feature by using the first correction function specific to first period t1 and corrects the second feature by using the second correction function specific to second period t2.

The amount of moisture in the sample gas adsorbed by odor sensor 8 gradually increases from the start of exposure to the sample gas. Due to such a temporal change in the amount of moisture adsorbed by odor sensor 8, the feature of the signal outputted from odor sensor 8 during first period t1 and the feature of the signal outputted from odor sensor 8 during second period t2 become different from each other.

In the present variation, by using the first correction function specific to first period t1 and the second correction function specific to second period t2, the features can be corrected with high accuracy while suppressing the influence of the temporal change in the amount of moisture adsorbed by odor sensor 8.

It should be noted that although second period T2 in measurement period Tm is divided into two periods (first period t1 and second period t2) in the present variation, the present disclosure is not limited to this example and second period T2 in measurement period Tm may be divided into three or more periods.

### 1-6. Variation 2

When a plurality of odor sensors 8 are provided, corrector 18 may use a plurality of correction functions each of which is specific to a corresponding one of the plurality of odor sensors 8. For example, when a first odor sensor (an example of a first sensor) and a second odor sensor (an example of a second sensor) are provided as a plurality of odor sensors 8, corrector 18 uses a first correction function specific to the first odor sensor and a second correction function specific to the second odor sensor.

In this case, signal obtainer 12 obtains a first signal outputted from the first odor sensor exposed to a sample gas during second period T2 (see FIG. 4) and obtains a second signal outputted from the second odor sensor exposed to the sample gas during second period T2.

Extractor 16 extracts a first feature from the first signal obtained by signal obtainer 12 and extracts a second feature from the second signal obtained by signal obtainer 12.

Then, corrector 18 corrects the first feature by using the first correction function and corrects the second feature by using the second correction function.

Thus, even when the first odor sensor and the second odor sensor are different in characteristics (e.g., responsivity to humidity or the like), the features can be corrected with high accuracy by using the correction functions each of which is optimal for a corresponding one of the first odor sensor and the second odor sensor.

It should be noted that although the first odor sensor and the second odor sensor are provided as a plurality of odor sensors 8 in the present variation, the present disclosure is not limited to this example and three or more odor sensors may be provided.

### 1-7. Variation 3

When extractor 16 extracts, as features, both the sensitivity and gradient of a signal, corrector 18 may use a first correction function specific to the sensitivity of the signal and a second correction function specific to the gradient of the signal. In this case, corrector 18 corrects the sensitivity of the signal by using the first correction function and corrects the gradient of the signal by using the second correction function.

Thus, even when the sensitivity and gradient of the signal are different in responsivity to humidity, the features can be corrected with high accuracy by using the correction functions each of which is optimal for a corresponding one of the features.

### Embodiment 2

### 2-1. Configuration of Gas Identification System

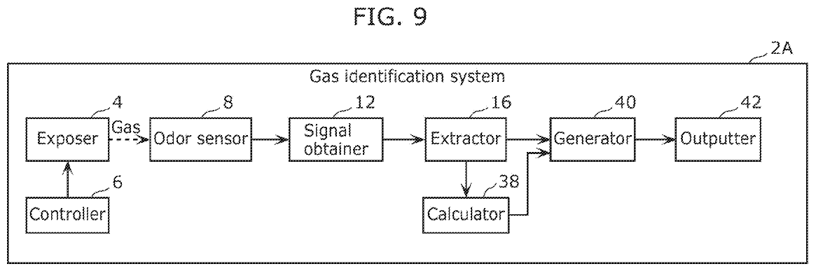

The configuration of gas identification system 2A according to Embodiment 2 is described with reference to FIG. 9. FIG. 9 is a block diagram illustrating the configuration of gas identification system 2A according to Embodiment 2. It should be noted that, in the present embodiment, the constituent elements that are the same as in Embodiment 1 share like reference signs, and description of such constituent elements is omitted.

As illustrated in FIG. 9, gas identification system 2A according to Embodiment 2 includes exposer 4, controller 6, odor sensor 8, signal obtainer 12, extractor 16, calculator 38, generator 40, and outputter 42. It should be noted that gas identification system 2A does not include humidity sensor 10, humidity data obtainer 14, corrector 18, storage 20, and identifier 22 that have been described in Embodiment 1.

Signal obtainer 12 obtains a signal outputted from odor sensor 8 exposed to a sample gas having a certain humidity (40%, for example) during second period T2 in measurement period Tm (see FIG. 4).

Calculator 38 calculates correction coefficient a based on a feature extracted by extractor 16, and outputs, to generator 40, correction coefficient a calculated.

Generator 40 generates, from the feature extracted by extractor 16, pseudo data sets indicating features corresponding to the sample gas having different humidities (0%, 20%, 60%, and 80%, for example) other than the certain humidity, based on correction coefficient a calculated by calculator 38. Generator 40 outputs, to outputter 42, the pseudo data sets generated.

Outputter 42 outputs, as training data sets to be used in a trained model for identifying the sample gas, the pseudo data sets generated by generator 40. It should be noted that outputter 42 outputs the training data sets to a storage (not illustrated) in which the trained model is stored. The storage may be disposed in gas identification system 2A or outside of gas identification system 2A (e.g., in a cloud server or the like).

### 2-2. Operations Performed by Gas Identification System

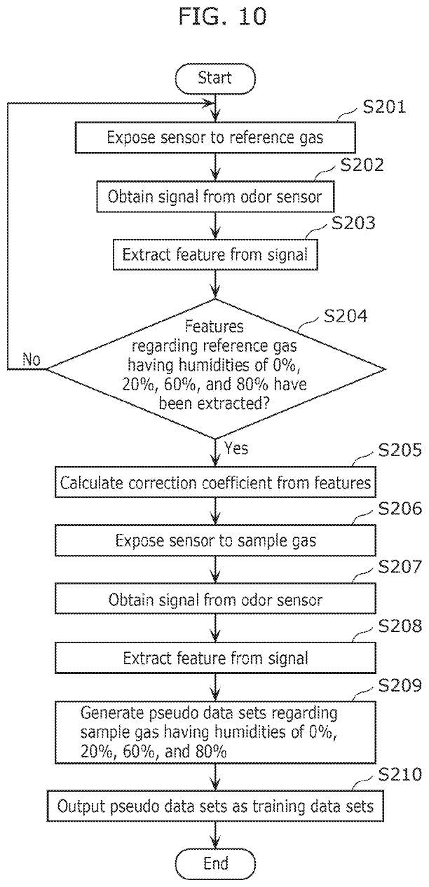

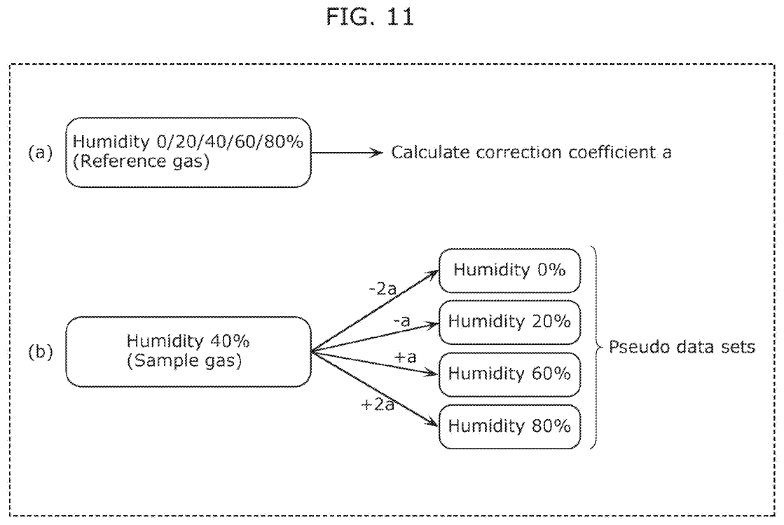

Next, operations performed by gas identification system 2A according to Embodiment 2 are described with reference to FIG. 10 and FIG. 11. FIG. 10 is a flowchart illustrating a flow of operations performed by gas identification system 2A according to Embodiment 2. FIG. 11 is a conceptual diagram for describing operations performed by gas identification system 2A according to Embodiment 2.

As illustrated in FIG. 10, controller 6 drives pump 28 (see FIG. 2) and switches three-way electromagnetic valve 26 (see FIG. 2) to the second state. Thus, a reference gas having the humidity of 0% is introduced into housing 24 (see FIG. 2) and odor sensor 8 is exposed to the reference gas having the humidity of 0% (S201). For example, the reference gas is nitrogen.

After that, signal obtainer 12 obtains a signal outputted from odor sensor 8 (S202) and outputs the signal obtained to extractor 16. Extractor 16 extracts a feature from the signal obtained by signal obtainer 12 (S203) and outputs the feature extracted to calculator 38.

When features regarding the reference gas having humidities of 0%, 20%, 60%, and 80% have not yet been extracted (S204: NO), steps S201 to S203 are performed again. In other words, steps S201 to S203 are repeatedly performed until features regarding the reference gas having humidities of 0%, 20%, 60%, and 80% have been extracted. When features regarding the reference gas having humidities of 0%, 20%, 60%, and 80% have been extracted (S204: YES), calculator 38 calculates correction coefficient a, based on the features extracted regarding the reference gas having humidities of 0%, 20%, 60%, and 80%, as illustrated in (a) of FIG. 11 (S205). Specifically, calculator 38 calculates, as correction coefficient a, the gradient of a linear function (y=ax+b) by which a function representing the relationship between the humidity and feature of the reference gas is approximated using the least-squares method.

Next, controller 6 drives pump 28 and switches three-way electromagnetic valve 26 to the first state. Thus, a sample gas having the humidity of 40%, for example, is introduced into housing 24 and odor sensor 8 is exposed to the sample gas having the humidity of 40% (S206).

After that, signal obtainer 12 obtains a signal outputted from odor sensor 8 (S207) and outputs the signal obtained to extractor 16. Extractor 16 extracts a feature from the signal obtained by signal obtainer 12 (S208) and outputs the feature extracted to generator 40.

Generator 40 generates, from the feature extracted regarding the sample gas having the humidity of 40%, pseudo data sets indicating features corresponding to the sample gas having humidities of 0%, 20%, 60%, and 80%, by using correction coefficient a calculated by calculator 38 (S209).

Specifically, as illustrated in (b) of FIG. 11, generator 40: (i) generates a pseudo data set indicating a feature corresponding to the sample gas having the humidity of 0%, by adding “−2a” to the feature extracted regarding the sample gas having the humidity of 40%; (ii) generates a pseudo data set indicating a feature corresponding to the sample gas having the humidity of 20%, by adding “−a” to the feature extracted regarding the sample gas having the humidity of 40%; (iii) generates a pseudo data set indicating a feature corresponding to the sample gas having the humidity of 60%, by adding “+a” to the feature extracted regarding the sample gas having the humidity of 40%; and (iv) generates a pseudo data set indicating a feature corresponding to the sample gas having the humidity of 80%, by adding “+2a” to the feature extracted regarding the sample gas having the humidity of 40%.

After that, outputter 42 outputs, as training data sets, the pseudo data sets generated by generator 40 (S210). Accordingly, for example, a trained model uses, in addition to a training data set corresponding to the humidity of 40% of the sample gas, the training data sets each of which corresponds to a different one of the humidities of 0%, 20%, 60%, and 80% of the sample gas.

### 2-3. Effect

In the present embodiment, the identification accuracy for a sample gas can be improved by using a pseudo data set as a training data set.

### 2-4. Example and Comparative Example

The following experiment was carried out for confirming the above-described effect. In this experiment, nitrogen was used as a reference gas. Moreover, as sample gases, sample gases A to E with five types of reference odors for odor determination (odor intensity level 2) were used. Sample gases A to E were gases each of which includes a different one of the five types of chemical compounds below.
Sample gas A: β-phenylethyl alcohol (odor of flower)
Sample gas B: Methylcyclopentenolone (odor of something sweet and burnt)
Sample gas C: Isovaleric acid (odor of sweaty socks)
Sample gas D: γ-Undecalactone (odor of ripe fruit)
Sample gas E: skatole (odor of mold)

As a comparative example, signals outputted from an odor sensor were obtained by exposing the odor sensor to sample gases A to E (at the temperature of 23° C.) having humidities of 0%, 20%, 40%, 60%, and 80%. A signal outputted from the odor sensor was obtained 100 times for each of sample gases A to E having the humidity of 0%. Moreover, a signal outputted from the odor sensor was obtained 100 times for each of sample gases A to E having the humidity of 20%. Furthermore, a signal outputted from the odor sensor was obtained 100 times for each of sample gases A to E having the humidity of 40%. Furthermore, a signal outputted from the odor sensor was obtained 100 times for each of sample gases A to E having the humidity of 60%. Furthermore, a signal outputted from the odor sensor was obtained 100 times for each of sample gases A to E having the humidity of 80%. A test for identifying a sample gas was performed by inputting, to a trained model, features extracted from the signals. It should be noted that a learned humidity learned by the trained model was 40%.

Moreover, as an example, signals outputted from an odor sensor were obtained by exposing the odor sensor to sample gases A to E (at the temperature of 23° C.) having humidities of 0%, 20%, 40%, 60%, and 80%. A signal outputted from the odor sensor was obtained 100 times for each of sample gases A to E having the humidity of 0%. Moreover, a signal outputted from the odor sensor was obtained 100 times for each of sample gases A to E having the humidity of 20%. Furthermore, a signal outputted from the odor sensor was obtained 100 times for each of sample gases A to E having the humidity of 40%. Furthermore, a signal outputted from the odor sensor was obtained 100 times for each of sample gases A to E having the humidity of 60%. Furthermore, a signal outputted from the odor sensor was obtained 100 times for each of sample gases A to E having the humidity of 80%. A test for identifying a sample gas was performed by inputting, to a trained model, features extracted from the signals. It should be noted that learned humidities learned by the trained model were 0%, 20%, 40%, 60%, and 80%. Among the learned humidities, learned humidities of 0%, 20%, 60%, and 80% were learned humidities corresponding to the training data sets generated from the above-described pseudo data sets.

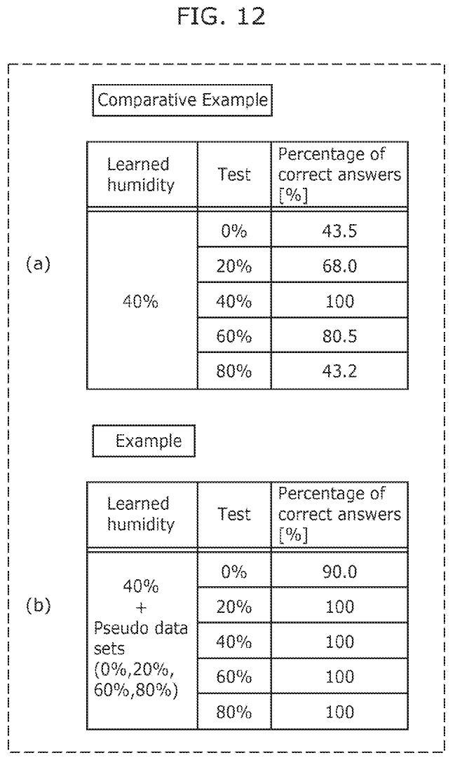

The result of the experiment is shown in FIG. 12. FIG. 12 is a table showing the result of the experiment for confirming an effect achieved by gas identification system 2A according to Embodiment 2.

As shown in (a) of FIG. 12, in the comparative example, the percentages of correct answers given by the trained model were 43.5% for sample gases A to E having the humidity of 0%, 68.0% for sample gases A to E having the humidity of 20%, 100% for sample gases A to E having the humidity of 40%, 80.5% for sample gases A to E having the humidity of 60%, and 43.2% for sample gases A to E having the humidity of 80%.

In contrast, as shown in (b) of FIG. 12, in the example, the percentages of correct answers given by the trained model were 90.0% for sample gases A to E having the humidity of 0%, 100% for sample gases A to E having the humidity of 20%, 100% for sample gases A to E having the humidity of 40%, 100% for sample gases A to E having the humidity of 60%, and 100% for sample gases A to E having the humidity of 80%.

Thus, it was confirmed that the percentages of correct answers given by the trained model in the example was increased compared to those in the comparative example.

### 2-5. Others

By outputting one or more pseudo data sets as one or more training data sets, a trained model is trained using two or more levels. In this case, the identification accuracy for a sample gas improves to a greater extent with training using two or more levels compared to with training using one level.

The following experiment was carried out for confirming the above-described effect. In this experiment, nitrogen was used as a reference gas. Moreover, as sample gases, sample gases A to E with five types of reference odors for odor determination (odor intensity level 2) were used. Sample gases A to E were gases each of which includes a different one of the five types of chemical compounds below.
Sample gas A: β-phenylethyl alcohol (odor of flower)
Sample gas B: Methylcyclopentenolone (odor of something sweet and burnt)
Sample gas C: Isovaleric acid (odor of sweaty socks)
Sample gas D: γ-Undecalactone (odor of ripe fruit)
Sample gas E: skatole (odor of mold)

As comparative example 1, signals outputted from an odor sensor were obtained by exposing the odor sensor to sample gases A to E (at the temperature of 23° C.) having the humidity of 40%. A signal outputted from the odor sensor was obtained 100 times for each of sample gases A to E. A test for identifying a sample gas was performed by inputting, to a trained model, features extracted from the signals. It should be noted that a learned humidity learned by the trained model was 20%.

Moreover, as comparative example 2, signals outputted from an odor sensor were obtained by exposing the odor sensor to sample gases A to E (at the temperature of 23° C.) having the humidity of 40%. A signal outputted from the odor sensor was obtained 100 times for each of sample gases A to E. A test for identifying a sample gas was performed by inputting, to a trained model, features extracted from the signals. It should be noted that a learned humidity learned by the trained model was 80%.

Furthermore, as an example, signals outputted from an odor sensor were obtained by exposing the odor sensor to sample gases A to E (at the temperature of 23° C.) having the humidity of 40%. A signal outputted from the odor sensor was obtained 100 times for each of sample gases A to E. A test for identifying a sample gas was performed by inputting, to a trained model, features extracted from the signals. It should be noted that learned humidities learned by the trained model were 20% and 80%. In other words, the humidity of 40% of sample gases A to E was a value between the learned humidities of 20% and 80%.

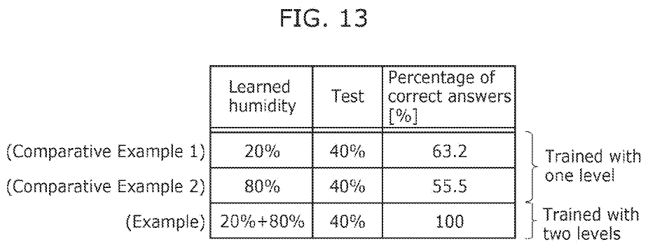

The result of the experiment is shown in FIG. 13. FIG. 13 is a table showing the result of the experiment for confirming an effect achieved by gas identification system 2A according to Embodiment 2.

As shown in FIG. 13, the percentage of correct answers given by the trained model was 63.2% in comparative example 1, and the percentage of correct answers given by the trained model was 55.5% in comparative example 2. In contrast, the percentage of correct answers given by the trained model was 100% in the example.

Thus, it was confirmed that, even for an unlearned humidity (40%), the identification accuracy for a sample gas improved to a greater extent with training using two or more levels compared to with training using one level.

### Other Variations

Although a gas identification method and a gas identification system according to one or more aspects have been described based on the above-described embodiments, the present disclosure is not limited to the above-described embodiments. Various modifications of the above-described embodiments as well as embodiments resulting from arbitrary combinations of constituent elements of different embodiments that may be conceived by those skilled in the art are intended to be included within the scope of the one or more aspects as long as they do not depart from the essence of the present disclosure.

Although signal obtainer 12 directly obtains a signal outputted from odor sensor 8 in the above-described embodiments, the present disclosure is not limited to this example. For example, signal obtainer 12 may obtain a signal outputted from odor sensor 8 via a network. In this case, odor sensor 8 may be disposed outside gas identification system 2 (2A).

Although gas identification system 2 includes storage 20 in Embodiment 1, the present disclosure is not limited to this example and storage 20 may be disposed outside gas identification system 2 (e.g., in a cloud server or the like).

Moreover, Embodiment 1 and Embodiment 2 may be combined. In other words, gas identification system 2 according to Embodiment 1 may further include calculator 38, generator 40, and outputter 42 that have been described in Embodiment 2.

It should be noted that, in each of the above-described embodiments, each of the constituent elements may be configured as dedicated hardware or may be realized by executing a software program suitable for the constituent element. Each of the constituent elements may be realized by a program executing unit, such as a CPU or a processor, reading and executing a software program recorded on a recording medium such as a hard disk or semiconductor memory.

Moreover, a part or all of the functions of each of the gas identification systems according to the above-described embodiments may be realized by a processor, such as a CPU, executing a program.

A part or all of the constituent elements of each of the above-described devices may be configured as an IC card which can be attached to and detached from the respective devices or as a stand-alone module. The IC card or the module is a computer system configured from a microprocessor, a ROM, and a RAM, for example. The IC card or the module may include a super-multifunctional LSI. The IC card or the module achieves its function through the microprocessor's operation according to the computer program. The IC card or the module may be tamper-resistant.

The present disclosure may be realized as the above-described method. Moreover, the present disclosure may be a computer program for executing the above-described method using a computer, or may be a digital signal generated by the computer program. Furthermore, the present disclosure may also be realized as a non-transitory computer-readable recording medium, such as a flexible disk, hard disk, CD-ROM, MO, DVD, DVD-ROM, DVD-RAM, Blu-ray (registered trademark) disc (BD), semiconductor memory, or the like, having recorded thereon the computer program or the digital signal. Furthermore, the present disclosure may include the digital signal recorded on the recording medium. Furthermore, the present disclosure may be realized by transmitting the computer program or the digital signal via, for example, an electric communication line, a wireless or wired communication line, a network such as the Internet, or data broadcasting. Furthermore, the present disclosure may be a computer system including memory storing the computer program and a microprocessor that operates according to the computer program. Furthermore, the present disclosure may be realized by another independent computer system by transmitting, to the independent computer system, the program or the digital signal recorded on the recording medium, or by transmitting, to the independent computer system, the program or the digital signal via the network or the like.

### INDUSTRIAL APPLICABILITY

A gas identification method according to the present disclosure is useful for a system or the like for identifying an odorant included in a sample gas, for example.

### REFERENCE SIGNS LIST

2, 2A gas identification system
4 exposer
6 controller
8 odor sensor
10 humidity sensor
12 signal obtainer
14 humidity data obtainer
16 extractor
18 corrector
20 storage
22 identifier
24 housing
26 three-way electromagnetic valve
28 pump
30a, 30b, 30c, 30d, 30e pipe
32 first inlet port
34 second inlet port
36 outlet port
38 calculator
40 generator
42 outputter

1. A gas identification method using a sensor that outputs a signal according to an adsorption concentration of a gas, the gas identification method comprising:
(a) obtaining a signal outputted from the sensor exposed to a sample gas during a predetermined measurement period;
(b) extracting a feature of the signal obtained in (a);
(c) obtaining humidity data indicating a humidity of the sample gas;
(d) correcting the feature extracted in (b), based on the humidity data obtained in (c); and
(e) identifying the sample gas by using a trained model for identifying the sample gas, based on the feature corrected in (d), and outputting an identification result.

2. The gas identification method according to claim 1, wherein
in (d), the feature extracted in (b) is corrected by using a correction function representing a relationship between the feature extracted in (b), the humidity data obtained in (c), and a reference humidity that is a humidity of the sample gas learned by the trained model.

3. The gas identification method according to claim 2, wherein
the correction function is represented by
$$Y = X + A ( {H}_{0} - H )$$
where
A represents a correction coefficient calculated from a gradient of a linear function by which a function representing a relationship between the humidity of the sample gas and the feature of the sample gas is approximated,
X represents the feature extracted in (b),
H represents the humidity data obtained in (c),
H₀ represents the reference humidity, and
Y represents a correction value of the feature extracted in (b).

4. The gas identification method according to claim 2, wherein
the predetermined measurement period includes at least a first period and a second period,
the correction function includes at least a first correction function specific to the first period and a second correction function specific to the second period,
in (b), a first feature of the signal outputted from the sensor exposed to the sample gas during the first period and a second feature of the signal outputted from the sensor exposed to the sample gas during the second period are extracted, and
in (d), the first feature extracted in (b) is corrected by using the first correction function and the second feature extracted in (b) is corrected by using the second correction function.

5. The gas identification method according to claim 2, wherein
the sensor includes at least a first sensor and a second sensor,
the correction function includes at least a first correction function specific to the first sensor and a second correction function specific to the second sensor,
in (a), a first signal outputted from the first sensor exposed to the sample gas during the predetermined measurement period and a second signal outputted from the second sensor exposed to the sample gas during the predetermined measurement period are obtained,
in (b), a first feature of the first signal obtained in (a) and a second feature of the second signal obtained in (a) are extracted, and
in (d), the first feature extracted in (b) is corrected by using the first correction function and the second feature extracted in (b) is corrected by using the second correction function.

6. The gas identification method according to claim 2, wherein
the feature includes a value of the signal and a gradient of the signal that have changed due to the sensor being exposed to the sample gas,
the correction function includes a first correction function specific to the value of the signal and a second correction function specific to the gradient of the signal,
in (b), the value of the signal and the gradient of the signal are extracted as the feature of the signal, and
in (d), the value of the signal is corrected by using the first correction function and the gradient of the signal is corrected by using the second correction function.

7. The gas identification method according to claim 1, wherein
in (b), the feature responsive to humidity is extracted.

8. The gas identification method according to claim 7, wherein
the feature includes at least one of a value of the signal or a gradient of the signal that have changed due to the sensor being exposed to the sample gas.

9. The gas identification method according to claim 1, wherein
in (a), the signal outputted from the sensor is obtained via a network.

10. The gas identification method according to claim 1, further comprising:
(f) generating a training data set to be used in the trained model based on the feature extracted in (b), and outputting the training data set generated.

11. The gas identification method according to claim 10, wherein
in (f), a plurality of training data sets each of which corresponds to a different one of a plurality of humidities of the sample gas are generated, and the plurality of training data sets generated are outputted.

12. A gas identification system comprising:
a sensor that outputs a signal according to an adsorption concentration of a gas;
an exposer that exposes the sensor to a sample gas during a predetermined measurement period;
a signal obtainer that obtains a signal outputted from the sensor during the predetermined measurement period;
an extractor that extracts a feature of the signal obtained by the signal obtainer;
a humidity data obtainer that obtains humidity data indicating a humidity of the sample gas;
a corrector that corrects the feature extracted by the extractor based on the humidity data obtained by the humidity data obtainer; and
an identifier that identifies the sample gas by using a trained model for identifying the sample gas, based on the feature corrected by the corrector, and outputs an identification result.

13. A gas identification method using a sensor that outputs a signal according to an adsorption concentration of a gas, the gas identification method comprising:
(a) obtaining a signal outputted from the sensor exposed to a sample gas having a certain humidity;
(b) extracting a feature of the signal obtained in (a);
(c) generating, from the feature extracted in (b), a pseudo data set indicating a feature corresponding to the sample gas having a humidity other than the certain humidity, based on a predetermined correction coefficient; and
(d) outputting, as a training data set to be used in a trained model for identifying the sample gas, the pseudo data set generated in (c).
